(19) United States
(12) Patent Application Publication
(10) Pub. No.: US 2011/0208247 A1
(43) Pub. Date: Aug. 25, 2011

(54) Bone Plate Screw Holes Convertible to Hooks

(76) Inventor: Abhishek Modi, West Chester, PA (US)

(21) Appl. No.: 13/033,320

(22) Filed: Feb. 23, 2011

Related U.S. Application Data
(60) Provisional application No. 61/308,071, filed on Feb. 25, 2010.

(51) Int. Cl. A61B 17/80

(52) U.S. Cl. 606/286

(57) ABSTRACT

A system for treating a bone, comprises a bone plate extending longitudinally from a first end to a second end and including a plurality of openings extending therethrough and a first hook member including a head sized and shaped to be lockingly received within a first one of the openings, the first hook member further including a spiked portion extending distally from the head to a sharp bone engaging distal end which, when the head is lockingly received within the first opening, projects distally from the bone plate toward a first target portion of bone to be engaged thereby to temporarily maintain the first target of bone in a desired spatial relation to the bone plate.

### PRIORITY CLAIM

The present application claims priority to U.S. Provisional Application Ser. No. 61/308,071 filed on Feb. 25, 2010 and entitled “Bone Plate Screw Holes Convertible to Hooks,” the entire disclosure of which is incorporated herein by reference.

### FIELD OF THE INVENTION

The present invention relates to devices for treating fractures and, in particular, to a device including a spiked tip to be used in combination with a bone plate to facilitate reduction of a fracture.

### BACKGROUND

Rigid plates are often utilized to stabilize damaged or weakened portions of bone, for example, at fracture sites. These bone plates generally include openings through which bone fixation elements are inserted into the bone to anchor the plates to target portions of bone. In some situations, however, small bone fragments are difficult to maneuver making compression across the damaged or weakened portion of the bone difficult to maintain while inserting bone fixation elements therethrough.

### SUMMARY OF THE INVENTION

The present invention is directed to a system for treating a bone, comprising a bone plate extending longitudinally from a first end to a second end and including a plurality of openings extending therethrough and a first hook member including a head sized and shaped to be lockingly received within a first one of the openings, the first hook member further including a spiked portion extending distally from the head to a sharp bone engaging distal end which, when the head is lockingly received within the first opening, projects distally from the bone plate toward a first target portion of bone to be engaged thereby to temporarily maintain the first target of bone in a desired spatial relation to the bone plate.

The present invention is further directed to a method, comprising lockingly coupling a first hook member within a first hole through a bone plate so that a spiked distal end of the first hook member projects distally from a bone facing surface of the bone plate and engaging the spiked distal end of the first hook member with a first target portion of bone to temporarily maintain the first target portion of bone in a desired spatial relation to the bone plate in combination with inserting a first bone fixation element into a second target portion of bone via a second hole through the bone plate to permanently secure the second target portion of bone to the bone plate and removing the first hook member from the first hole after the first bone fixation element has secured the second target portion of bone to the bone plate.

### BRIEF DESCRIPTION OF THE DRAWINGS

FIG. 1 shows a perspective view of a system according to an exemplary embodiment of the present invention;

FIG. 2 shows a side view of the system of FIG. 1;

FIG. 3 shows a side view of a spike of the system of FIG. 1; and

FIG. 4 shows a top view of the spike of FIG. 3.

### DETAILED DESCRIPTION

The present invention may be further understood with reference to the following description and the appended drawings, wherein like elements are referred to with the same reference numerals. The present invention relates to devices for treating fractures. In particular, exemplary embodiments of the present invention describe a device including a spiked tip to be used in combination with a bone plate to facilitate reduction of a fracture. It will be understood by those of skill in the art that the terms proximal and distal, as used herein, are used to refer to a direction toward (proximal) and away from (distal) a user (e.g., surgeon) of the device.

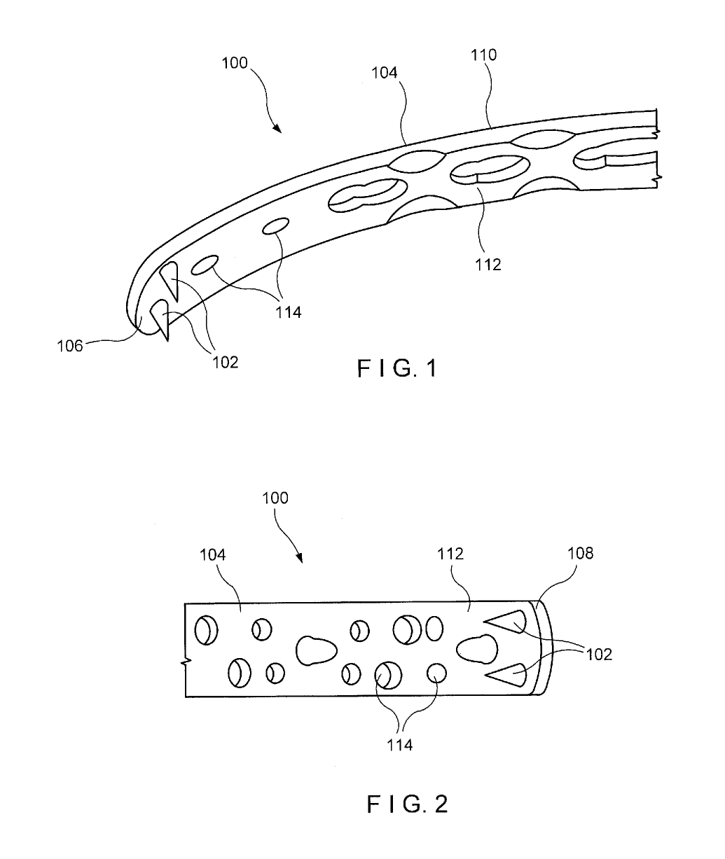

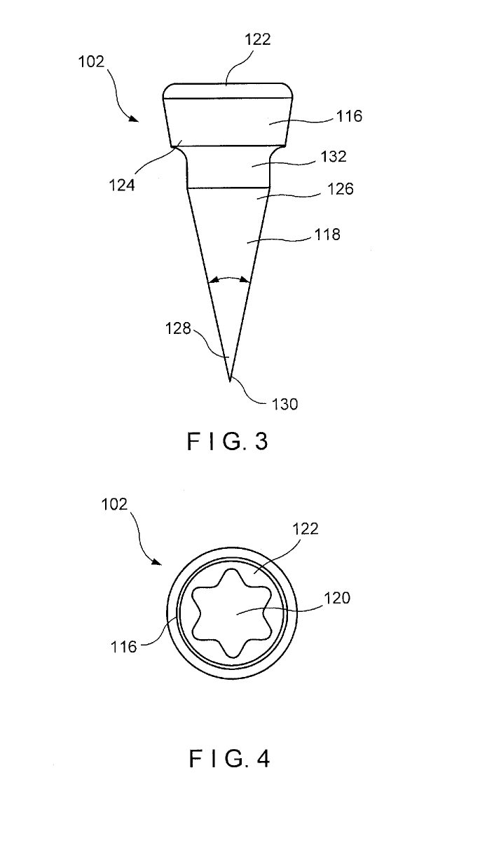

As shown in FIGS. 1 and 2, a system 100 according to a first exemplary embodiment of the present invention comprises a hook member 102 couplable to a bone plate 104 to provide the bone plate 104 with hooks to facilitate reduction of a fracture. In some situations (e.g., involving small bone fragments) the fragments may be difficult to maneuver, thereby resulting in insufficient reduction of the fracture and/or difficulty maintaining the reduction. The hook member 102 facilitates a preliminary fixation, allowing compression across the fracture using the bone plate 104 as a tension band. The preliminary fixation permits bone fixation elements to be easily inserted into the bone through the bone plate 104 to anchor the bone plate 104 to the bone while maintaining compression of the fracture. In an exemplary embodiment, the system 100 includes at least two hook members 102 at opposite ends of the bone plate 104. It will be understood by those of skill in the art, however, that the system 100 may include any number of hook members 102 and any number of locations at which they may be coupled to the bone plate 104 so long as the hook members 102 are positioned to engage target portions of the bone to facilitate a preliminary fixation of the bone.

The bone plate 104 extends longitudinally from a first end 106 to a second end 108 and includes a first surface 110 which, when in an operative position, faces away from the bone, and a second surface 112 which, when in an operative position, faces toward the bone. The bone plate 104 further includes a plurality of openings 114 extending therethrough, from the first surface 110 to the second surface 112. The openings 114 may be configured and adapted to receive the hook member 102 as well as bone fixation elements such as screws, nails and pins. The openings 114 may include an engagement mechanism such as, for example, a threading (not shown) along an inner surface thereof for engaging a portion of the hook member 102. The bone plate 104 may be an olecranon plate or a distal humerus plate. It will be understood by those of skill in the art, however, that the bone plate 104 may be any kind of bone plate that may be used to fix fractures. For this purpose, it is preferred to use standard locking holes configured to lockingly engage the heads of standard bone fixation elements such as screw and pins.

The hook member 102 includes a head portion 116 and a spiked portion 118 extending distally therefrom. The head portion 116 is sized and shaped to be received within a hook member opening 114 extending through the bone plate 104. The head portion 116 may include an engaging element such as, for example, threading (not shown) extending around an outer surface thereof to engage corresponding threading in the opening 114. As would be understood by those skilled in the art, if the openings 114 are standard locking holes, the head portion 116 will preferably be formed substantially similarly to the head portion of a bone fixation element to be coupled therein. The head portion 116 may further include a driving element 120 at a proximal end 122 thereof sized and shaped to engage a portion of a driving tool used to rotate the hook member 102 into threaded engagement with the opening 114 as would be understood by those skilled in the art to releasably fix the hook member 102 to the bone plate 104. The driving element 120 may be a recess at the proximal end 122 sized and shaped to receive a correspondingly shaped portion of the driving tool. For example, the driving element 120 may be a hexagonally shaped recess for engaging a hex-shaped portion of the driving tool. Alternatively, the driving element 120 may be a protrusion extending from the proximal end 122, the protrusion being sized and shaped to engage the driving tool.

The spiked portion 118 may be attached to a distal end 124 of the head portion 116 via a neck portion 132 with the spiked portion 118 extending distally therefrom. A diameter of the neck portion 132 in this embodiment is smaller than a diameter of the head portion 116 with the spiked portion 118 tapering from a proximal end 126 thereof (at the distal end of the neck portion 132) to a distal end 128 which ends in a sharp tip 130. The angle of this taper preferably ranges between approximately 15° and 45°. However, the taper angle will vary depending on a number of factors such as, for example, a desired length of the hook member 102, a desired strength of the hook member 102 and a hardness of the bone being treated. A length of the head portion 116 and the neck portion 132 is selected so that, when the hook member 102 is fully engaged with the opening 114, the spiked portion 118 extends distally from the second surface 112 of the bone plate 104 toward the portion of bone it is to engage. The sharp tip 130 is pressed into this target portion of bone to hold the bone plate 104 and this target portion of bone in a desired spatial relationship to one another, at least until the plate 104 is permanently coupled to the various portions of the bone. The sharp tip 130 may be impacted into the bone and/or engaged with a pre-drilled hole in the bone.

In a preferred embodiment, the system 100 includes at least two hook members 102, with each hook member 102 inserted into a corresponding opening 114. For example, a first one of the hook members 102 may be inserted into an opening 114 at a first end 106 of the plate 104 while a second one of the hook members 102 is inserted into an opening 114 at a second end 108 of the plate 104. The bone plate 104 according to this embodiment includes a plurality of openings 114 positioned to enable a user to mount a desired number of the hook members 102 at positions on the plate 104 corresponding to a plurality of target portions of bone to be temporarily held in place. Openings 114 are included at each of the first and second ends 106, 108, for example, to allow the bone plate 104 to be coupled across a fracture site via hook members 102 to preliminarily fix a portion of the bone prior to the insertion of permanent bone fixation elements (e.g., bone screws, pins, etc.). It will be understood by those of skill in the art, however, that the openings 114 may be positioned at any desired locations on the plate 104 suitable to facilitate engagement of hook members 102 with target portions of the bone.

A method according to the present invention may include preassembly of the hook member 102 with the bone plate 104 prior to a procedure. For example, any number of hook members 102 may be inserted into a corresponding openings 114 and coupled to the plate 104 as described above via, for example, rotation about central axes of the openings 114 to engage threads of the head portions 116 with threads of the openings 114. The plate 104 may then be placed on the bone over target portions or fragments thereof in a known manner. The user then engages the target portions of bone with the corresponding hook members 102 to temporarily hold the bone fragments in place. For example, the hook members 102 may be impacted into the target portions of the bone using a bone tamp, a mallet and/or other impaction device. Alternatively, the bone may be pre-drilled with small holes corresponding to a position of each of the hook members 102 such that the sharp tips 130 engage the pre-drilled holes when the plate 104 is placed over and pressed into the bone. The user then places any desired number of conventional bone fixation elements (e.g., bone screws or pins) through other openings in the plate to permanently anchor the bone plate 104 to the bone. As would be understood by those skilled in the art, the bone plate 104 may include any number and variety of bone fixation receiving holes such as combination holes, compression holes, variable angle locking holes, etc.

Once the bone plate 104 has been appropriately anchored to the bone, the hook members 102 may be removed from the bone plate 104 via, for example, rotating the hook members 102 about the central axis of the opening in a direction opposite that used to couple the hook members 102 therein. Upon removal of each of the hook members 102, a bone fixation element may be inserted into the opening 114 vacated by the hook member 102 to provide additional fixation of the bone plate 104. Thus, it will be understood by those of skill in the art that an advantage of the system 100 is the removable coupling of the hook members 102 to the bone plate 104.

It will be apparent to those skilled in the art that various modifications and variations can be made in the structure and the methodology of the present invention, without departing from the spirit or scope of the invention. Thus, it is intended that the present invention cover the modifications and variations of the invention provided that they come within the scope of the appended claims and their equivalents.

What is claimed is:

1. A system for treating a bone, comprising:
a bone plate extending longitudinally from a first end to a second end and including a plurality of openings extending therethrough; and
a first hook member including a head sized and shaped to be lockingly received within a first one of the openings, the first hook member further including a spiked portion extending distally from the head to a sharp bone engaging distal end which, when the head is lockingly received within the first opening, projects distally from the bone plate toward a first target portion of bone to be engaged thereby to temporarily maintain the first target of bone in a desired spatial relation to the bone plate.

2. The system of claim 1, wherein the first opening includes an internal threading and is sized and shaped to threadedly engage a threaded head of a bone fixation element, the head of the first hook member being sized and shaped substantially similarly to the head of the bone fixation element and including a thread thereon configured to engage the threading of the first opening.

3. The system of claim 1, further comprising a second hook member including a head sized and shaped to be lockingly received within a second one of the openings, the second hook member further including a spiked portion extending distally from the head to a sharp bone engaging distal end which, when the head is lockingly received within the second opening, projects distally from the bone plate toward a second target portion of bone to be engaged thereby to temporarily maintain the second target of bone in a desired spatial relation to the bone plate and to the first target portion of bone.

4. The system of claim 3, wherein the second opening includes an internal threading and is sized and shaped to threadedly engage a threaded head of a bone fixation element, the head of the second hook member being sized and shaped substantially similarly to the head of the bone fixation element and including a thread thereon configured to engage the threading of the second opening.

5. The system of claim 1, wherein the sharp tip of the first hook member tapers at an angle between 15 and 45 degrees.

6. The system of claim 1, wherein a proximal end of the head portion of the first hook member includes a driving element configured to mate with a driving tool to drive the first hook member into the first opening and to withdraw the first hook member therefrom.

7. A method, comprising:
lockingly coupling a first hook member within a first hole through a bone plate so that a spiked distal end of the first hook member projects distally from a bone facing surface of the bone plate;
engaging the spiked distal end of the first hook member with a first target portion of bone to temporarily maintain the first target portion of bone in a desired spatial relation to the bone plate;
inserting a first bone fixation element into a second target portion of bone via a second hole through the bone plate to permanently secure the second target portion of bone to the bone plate; and
removing the first hook member from the first hole after the first bone fixation element has secured the second target portion of bone to the bone plate.

8. The method of claim 7, further comprising the steps of:
lockingly coupling a second hook member within a third hole through the bone plate so that a spiked distal end of the second hook member projects distally from the bone facing surface of the bone plate;
engaging the spiked distal end of the second hook member with a third target portion of bone to temporarily maintain the third target portion of bone in a desired spatial relation to the bone plate; and
removing the second hook member from the third hole after the first bone fixation element has secured the second target portion of bone to the bone plate.

9. The method of claim 8, further comprising the step of inserting a second bone fixation element into a fourth target portion of bone via a fourth hole through the bone plate to permanently secure the fourth target portion of bone to the bone plate, wherein the first and second target portions of bone are on a first side of a fracture in the bone and the third and fourth target portions of bone are on a second side of the fracture.

10. The method of claim 7, wherein engaging the spiked distal end of the first hook member with the first target portion of bone includes impacting the first hook member into the first target portion using an impaction device.

11. The method of claim 7, further comprising the step of drilling a first hole into the first target portion, a position of the first hole corresponding to a desired position of the first hook member.

12. The method of claim 11, wherein engaging the spiked distal end of the first hook member with the first target portion of bone includes engaging the first hook member with the first hole in the first target portion.

13. The method of claim 8, wherein engaging the spiked distal end of the of the second hook member with the third target portion of bone includes impacting the second hook member into the third target portion using an impaction device.

14. The method of claim 9, further comprising drilling a second hole into the third target portion, a position of the second hole corresponding to a desired position of the second hook member.

15. The method of claim 14, wherein engaging the spiked distal end of the second hook member with the third target portion of bone includes engaging the second hook member with the second hole in the third target portion.
